(19) United States
(12) Plant Patent Application Publication
(10) Pub. No.: US 2003/0014781 P1
(43) Pub. Date: Jan. 16, 2003

(54) Miniature rose plant named: 'Jentara'

(76) Inventor: Svend Jensen, Christiansfeld (DK)

Correspondence Address: Svend Jensen, Kongensgade 32, Christiansfeld, 6070, DK

(21) Appl. No.: 10/136,613

(22) Filed: May 2, 2002

Related U.S. Application Data
(63) Continuation-in-part of application No. 09/772,695, filed on Jan. 31, 2001, now abandoned.

(51) Int. Cl. A01H 5/00

(52) U.S. Cl. PLT/117

(57) ABSTRACT

A new miniature rose plant which has abundant, dark red colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

### BACKGROUND OF THE INVENTION

The present invention constitutes a new and distinct variety of miniature rose plant, which was developed by artificially pollinating an unnamed seedling(not patent in the US) with an unnamed seedling (not patent in the US). The two parents were crossed in the summer of 1996 and the resulting seed was sown in December, 1996 in a controlled glasshouse environment. Out of these seedlings one seedling was selected, as the new variety and named ‘Jentara’. The new rose may be distinguished from its seed parent, an unnamed seedling, by the following combination of characteristics:

1. The unnamed seedlings has a breeding background in unnamed seedlings.

2. ‘Jentara’ has small double flowers, while the unnamed seedling has big single flowers.

3. ‘Jentara’ has white coloured petals, while the unnamed seedling has creme white petals.

The new variety may distinguished from its pollen parent, an unnamed seedling created by the same inventor, by the following combination of characteristics:

1. The unnamed seedling has a breeding background in unnamed seedlings.

2. ‘Jentara’ has smaller flowers and foliage as compared to the unnamed seedling.

3. ‘Jentara’ has dark white coloured petals, while the unnamed seedling has pale yellow petals.

### BREIF SUMMARY OF THE INVENTION

Initial asexual reproduction of ‘Jentara’ by cuttings was first done in Bjert, Denmark The reproduction was conducted in controlled greenhouse environments.

Have here proven to be stabile by propagation with cuttings in several generations. ‘Jentara’ is a low and compact miniature rose with medium vigor. The shelflife has been tested to be good.

The objective of the hybridization of this rose variety for commercial greenhouse culture was to create a new and distinct variety with:

1. Uniform and abundant flowers with good keepability;

2. Attractive long lasting foliage and compact growth,

3. Year round flowering under glasshouse conditions;

4. Suitability for production from softwood cuttings in pots.;

5. Durable flowers and foliage which make the variety suitable for distribution in the floral industry.

This combination of qualities was not present in previously available commercial cultivars of this type and distinguish ‘Jentara’ from all other varieties of which we are aware.

The seeds from hybridization were planted in a controlled environment and evaluations were conducted on the resulting plants. ‘Jentara’ was selected by Svemd Jensen in he's development program in Bjert, Denmark.

### BREIF DESCRIPTION OF THE DRAWINGS

The accompanying color illustrations show as true as is reasonably to obtain in color photographs of this type, the typical characteristics of the buds, flowers, leaves, stems of ‘Jentara’. Specifically illustrated in: Photo SHEET 1: Young shoot.

FIG. 1. Bud before opening the sepals.

FIG. 2. Bud at the opening the sepals.

FIG. 3. Bud at the opening the petals.

FIG. 4. Flower during course of opening.

FIG. 5. Open flower—plan view—obverse.

FIG. 6. Open flower—plan view—reverse.

FIG. 7. Fully open flower—plan view—obverse.

FIG. 8. Fully open flower—plan view—reverse. Photo SHEET 2:

FIG. 9. Receptacle showing stamens and pistils.

FIG. 10. Receptacle showing pistils (stamens removed).

FIG. 11. Flower petals, detached—outer surface.

FIG. 12. Flower petals, detached—inner surface.

FIG. 13. Bare stem exhibiting thorns and flower attachment.

FIG. 14. Three leaflets upper side.

FIG. 15. Three leaflets reverse side.

FIG. 16. Five leaflets upper side.

FIG. 17. Five leaflets reverse side.

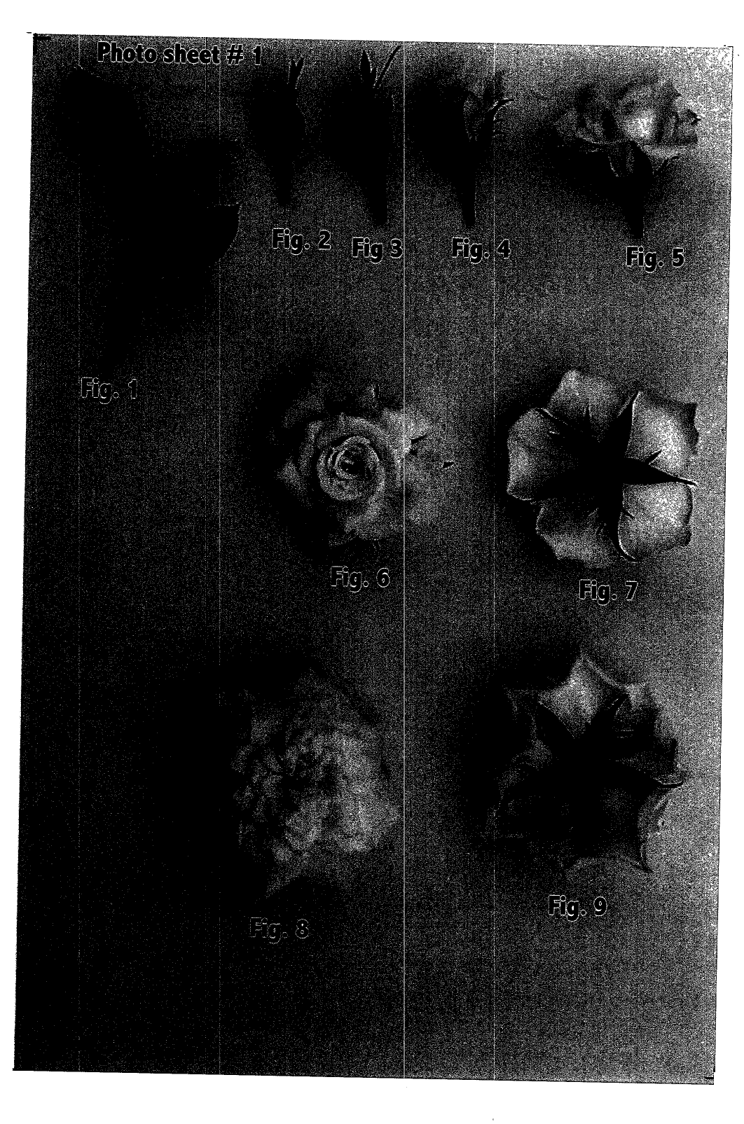

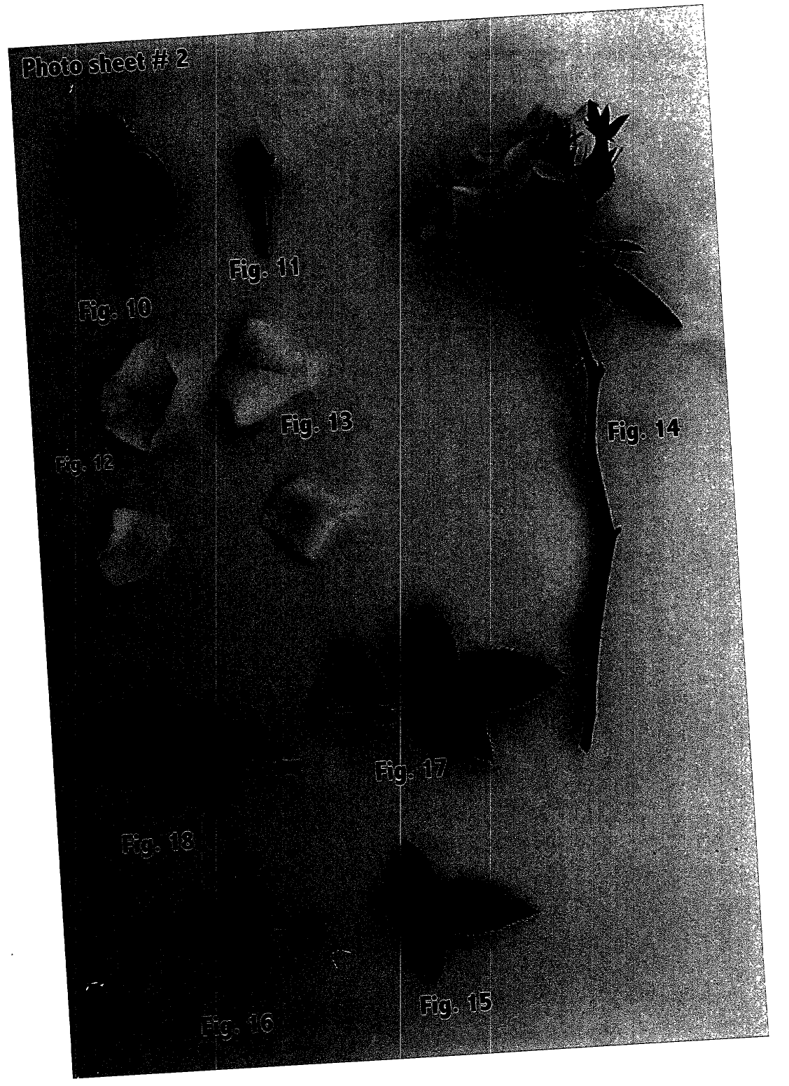

### DETAILED BOTANICAL DECRIPTION OF THE VARIETY

The following is a detailed description of the Miniature Rose: Rosa hybrid ‘Jentara’.

The following observations, measurements, values and comparisons describe plants grown in glass houses in Bjert, Denmark.

The age of the observed plants where 11 to 13 weeks after propagation by cuttings, and produced as flowering pot plants in container of 10.5 centimeter in diameter.

Color references are made using The Royal Horticultural Society (London, England) Colour Chart, 1995, except where common terms of color are used. For a comparison, the nearest existing rose variety is ‘JACtink’, a rose variety described and illustrated in U.S. Plant Pat. No. 113,98.

Chart 1 details several physical characteristics of ‘Jentara’ and ‘JACtink’.

CHART 1

| | ‘Jentara’ | ‘JACtink’ |
|---|---|---|
| Petal color, Upper | White Group 155C | White Group 155A |
| surface | | |
| Petal color, Reverse | White Group 155C | White Group 155A |
| surface | | |
| Petal count | 75 | 65 to 70 |

Parents: Unnamed seedling. Times. Unnamed seedling

Classification

Botanical.—Rosa hybrid

Commercial.—Miniature

Plant:

Plant growth.—Moderately vigorous. Grows compact upright to bushy. When grown as 10 cm pot plant, the average height of the plant itself is 18 to 20 cm, and average with is 20 cm. Production time is generally 11 to 13 weeks depending on average temperature, light level, and cultural practices.

Stem:

Color.—Young wood: Green Group 138B, with intonations of Greyed-Red Group 178B Older wood: Green Group 137A

Thorns.—Incidence: Low number of thorns Size: 7-8 mm Color: Red-Purple Group 57D Shape: Deep concave.

Surface.—Young wood: Smooth Older wood: Smooth

Stem diameter.—2 mm

Internode length.—10 -20 mm

Numbers of internodes.—7-8

PLANT FOLIAGE: Leaves arranged alternately, compound with three to seven leaflets per leaf, generally symmetrical, abundant, and flat in aspect. Stipules at petiole base. Quantity of leaves: 7 to 18 per lateral branch.

Leaf size.—Medium 50-75 mm(l). times. 38-45 mm(w)

Petioles.—Color:Green Group 137A, with intonations of Greyed-Red Group 178B Margins: entire Length: 15-25 mm Diameter: about 1-2 mm.

Stipules.—Size: 5-6 mm Surface: Smooth Colour: Green Group 137A, with intonations of Greyed-Red Group 178B Margins: entire

Rachis.—Color: Green Group 137A, with intonations of Greyed-Red Group 178B Margins: Margins with stipitate glands. Length: 10 to 20 mm

Leaflets.—Edge: Serrated Serration: Single. Venation pattern: Pinnate Shape: Ovate with acute apex and obtuse base

Texture.—Smooth

Appearance.—Glossy

Size.13 length: 20 to 50 mm. With: 12 to 30 mm

Color.—Young foliage: Upper surface: Green Group 146B, with intonations of Greyed-Red Group 178B Lower surface: Green Group 146B, with intonations of Greyed-Red Group 178B Color: Mature foliage: Upper surface: Green Group 137A Lower surface: Greyed-Green Group 138B

Inflorescense:

Blooming habit.—Recurrent

Number of flowers.—Generally 1 to 3 bud per flowering stem.

Penduncle.—Color: Green Group 137B, With intonations of Greyed-Red Group 178B Texture: Smooth, with pubescence and prickles Length: 35-50 mm Angle: Upright

Receptacle.—Surface: Smooth, glabrous. Shape: Funnel-shaped Size: h: 6-7 mm w: 5-6 mm Color:-Green Group 137B, with intonations of Greyed-Red Group 178B

Sepals.—Quantity: 5 Shape: Narrowly Ovate w. acute tip Texture: Leathery Margin: Foliaceous appendages on 3 of the five sepals Appearance: Dull Color: Upper surface: Green Group 137C Reverse surface: Green Group 138A

Buds.—Size: 20-25 mm (h) 12-15 mm (w)upon opening. Shape: Cupped Colour: at ¼ opening, White Group 155C

Flower.—

Duration.—As a pot plant, flowers last form 16 to 21 days. Fragrance. None. Size: 40-50 mm in diameter.

Form.—Shape of flower when viewed from the side. Up on opening: Cupped Open flower: Cupped Color: Petals, upon opening. Upper surface: White Group 1 55C, with stripe in middle of Green Group 138A Reverse surface: White Group 155C, with stripe in middle Of green Group 138A Petals after opening: Upper surface: White Group 155C. Reverse surface: White Group 155C. Basal Petals spots: Size: 1-2 mm Colour: Yellow-White Group 158B General tonality: On Open flower: Third day: White Group 155C Afterwards: White Group 155B

Petals.—

Petal reflex.—Outermost petals reflex backwards at opening. Fully open all petals reflex backwards.

Texture.—Smooth.

Petal edge.—Uniform

Petal count.—Approximately 75 on the average per flower.

Petal size.—Length 12-28 mm With: 8-25 mm.

Shape.—Outer petals: Round Inner petals: Ovate

Reproductive organs.—

Stamen number.—Approximately 10 on average per flower

Pollen.—Color: Yellow Group 13B, Abundance: Average

Anthers.—Size: 2-3 mm Color: Yellow Group 13B, Shape: Oblong.

Filaments.—Size: 4-5 mm Color: Green-White Group 157C.

Pistils number.—Approximately 30 on average per flower.

Stigmas.—Location: Superior in location to anthers Color: White Group 155C

Styles.—Color: White Group 155C, with intonations of Red-Purple Group 67A. Length: 2 to 3 mm

Development:

Vegetation.—Dense

Blooming.—Abundant.

Aptitude to bear fruit.—Poor.

Resistance to diseases.—below average resistance to mildew and Botrytis under normal growing conditions in Bjert, Denmark. Hips/seeds has not been observed due to that the plant has never been grown to the stage of seed development, due to the fact, that the variety is developed for use as a flowering pot plant only.

Winter hardiness & Drought/heat tolerance.—Due to the fact, that this variety is a potted flowering plant, developed for one time use only, the plant is not tested for winter hardiness or drought/heat tolerance

1. A new and distinct variety of rose plant of the miniature class, substantially as herein illustrated and described as a distinct and novel rose variety due to its abundant bronze-peach blend flowers, attractive long lasting foliage, vigorous and compact growth, year round flowering under glasshouse conditions, suitability for production from softwood cuttings in pots, and durable flowers and foliage which make the variety suitable for distribution in the floral industry.
